(19) United States
(12) Patent Application Publication
(10) Pub. No.: US 2018/0321684 A1
(43) Pub. Date: Nov. 8, 2018

(54) CONTROLLING MOVEMENT OF MEDICAL APPARATUS

(71) Applicant: Beijing Neusoft Medical Equipment Co., Ltd., Beijing (CN)

(72) Inventor: Shang GAO, Beijing (CN)

(21) Appl. No.: 15/971,972

(22) Filed: May 4, 2018

(30) Foreign Application Priority Data
May 5, 2017 (CN) 201710311855.X

(51) Int. Cl. G05D 1/02; G05D 1/00; A61B 6/00; A61B 34/30

(52) U.S. Cl. CPC G05D 1/0223; G05D 1/0088; G05D 1/0214; A61B 6/4405; Y10S 901/01; G05D 1/027; G05D 1/024; G05D 2201/0206; A61B 34/30

(57) ABSTRACT

A medical apparatus is provided. According to an example, the medical apparatus may include a gantry, a plurality of movable wheels disposed at a bottom of the gantry, and a movement controller; where each of the movable wheels comprises a hub and a plurality of driven wheels disposed at the periphery of the hub, an axis of each of the driven wheels and an axis of the hub forming an included angle and rotation of the hub driving rotation of the driven wheels, and the movement controller configured to control a moving direction and a moving speed of the gantry by controlling rotation of the plurality of the movable wheels.

### CROSS REFERENCE TO RELATED APPLICATIONS

This application claims priority to Chinese Patent Application No. 201710311855.X filed on May 5, 2017, the entire contents of which is incorporated herein by reference in its entirety for all purposes.

### TECHNICAL FIELD

The present disclosure relates to a method of controlling movement of a medical apparatus.

### BACKGROUND

With the continuous development in medical care, more and more medical apparatuses have been used to assist doctors in medical diagnosis or treatment. For example, an angiography machine may facilitate diagnosis and treatment by entering a blood vessel or tissue of a subject via a blood puncture path under the guide of X-rays.

NEUSOFT MEDICAL SYSTEMS CO., LTD. (NMS), founded in 1998 with its world headquarters in China, is a leading supplier of medical equipment, medical IT solutions, and healthcare services. NMS supplies medical equipment with a wide portfolio, including CT, Magnetic Resonance Imaging (MRI), digital X-ray machine, ultrasound, Positron Emission Tomography (PET), Linear Accelerator (LINAC), and biochemistry analyser. Currently, NMS's products are exported to over 60 countries and regions around the globe, serving more than 5,000 renowned customers. NMS's latest successful developments, such as 128 Multi-Slice CT Scanner System, Superconducting MRI, LINAC, and PET products, have led China to become a global high-end medical equipment producer. As an integrated supplier with extensive experience in large medical equipment, NMS has been committed to the study of avoiding secondary potential harm caused by excessive X-ray irradiation to the subject during CT scanning process.

### BRIEF DESCRIPTION OF DRAWINGS

FIG. 1 is a schematic diagram illustrating a medical apparatus according to an example of the present disclosure.

FIG. 2 is a front view illustrating a movable wheel of a medical apparatus according to an example of the present disclosure.

FIG. 3 is a left view illustrating a movable wheel of a medical apparatus according to an example of the present disclosure.

FIG. 4 is a module block diagram illustrating a medical apparatus according to an example of the present disclosure.

FIG. 5(1), FIG. 5(2), FIG. 5(3), FIG. 5(4), FIG. 5(5), FIG. 5(6), FIG. 5(7), FIG. 5(8), FIG. 5(9), FIG. 5(10), FIG. 5(11) and FIG. 5(12) are schematic diagrams illustrating driving modes of moving a gantry according to an example of the present disclosure.

FIG. 6 is a module block diagram illustrating a medical apparatus according to another example of the present disclosure.

FIG. 7 is a flowchart illustrating a method of controlling movement of a medical apparatus according to an example of the present disclosure.

FIG. 8 is a flowchart illustrating a block of controlling movement of a movable wheel the method of controlling movement of a medical apparatus shown in FIG. 7 according to an example of the present disclosure.

### DETAILED DESCRIPTION

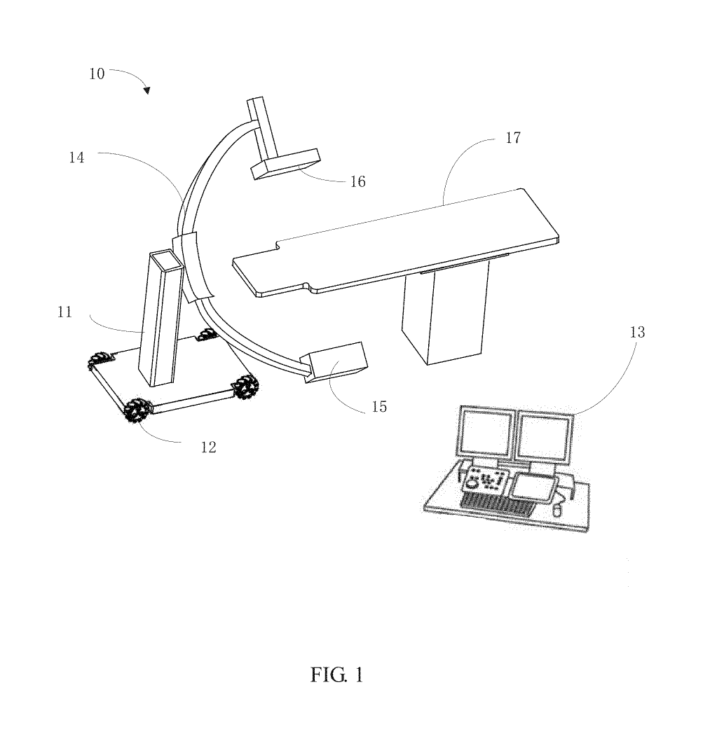

FIG. 1 is a schematic diagram illustrating a medical apparatus 10 according to an example of the present disclosure. The medical apparatus 10 may be an angiography machine. For example, the angiography machine may include a rotatable support arm (e.g., C-shaped arm) 14 provided with an imaging device, a gantry 11 capable of moving along a linear guide or rotating, a scanning bed 17, etc. With the rotation or linear movement of the gantry 11 or the rotation of the support arm 14, images of a blood vessel may be acquired at a plurality of angles or positions for a subject on the scanning bed 17 to assist a doctor in medical diagnosis or treatment. The medical apparatus 10 shown in FIG. 1 may include: a gantry 11, a plurality of movable wheels 12, a controller 13, a support arm 14, an X-ray source 15, a detector 16 and a scanning bed 17.

Gantry 11 may include a base and a column. The plurality of movable wheels 12 may be disposed at the bottom of the gantry 11, such as to the base of the gantry. The plurality of movable wheels 12 may be controlled to rotate in such a way that the gantry 11 may move with required or desired direction and speed. In an example shown in the figures, a number of the movable wheels 12 may be four, which is not limited herein. The movable wheels 12 may be symmetrically disposed at the bottom of the gantry 11. However, other numbers of movable wheels 12 may also be provided. The number of the movable wheels 12 may be set according to a practical application. In an example, the number of the movable wheels 12 may be an even number equal to or greater than 4 (including 4), such as 6, 8, etc. The gantry 11 may be more easily controlled by setting an even number of movable wheels 12. In an example, the number of the movable wheels 12 may be an even number equal to or greater than 6 (including 6), such as 10, 12, etc. With the number of movable wheels 12 being even and more than 6, the gantry 11 may be supported better, so that the gantry 11 may be more stable in a moving process. The movable wheels 12 may include, but are not limited to, mecanum wheels.

The support arm 14 is rotatably mounted on the gantry 11. In an example shown in the figures, the support arm 14 is a C-shaped arm. In another example, the support arm 14 may be a G-shaped arm. The X-ray source 15 and the detector 16 are oppositely disposed at two ends of the support arm 14. The X-ray source 15 and the detector 16 are positioned at opposite sides of a subject on the scanning bed 17 by the support arm 14. Thus, positions and angles of the X-ray source 15 and the detector 16 relative to the subject on the scanning bed 17 may be adjusted by controlling the movement of the gantry 11 and the rotation of the support arm 14.

The controller 13 may include a processor and a memory. The memory may be configured to store readable instructions such as a movement control. The processor may be configured to read the readable instructions stored in the memory to execute the movement control and so on. The memory may also be configured to store data such as a parameter, a moving path, an image and the like. The processor may also be configured to read data stored in the memory and perform relevant processing. The controller 13 may include one or more displays for displaying a moving path and/or a parameter, etc.

It is to be noted that the medical apparatus 10 described herein may be an angiography apparatus, for example, an angiography machine, and may also be other apparatuses for diagnosis and treatment.

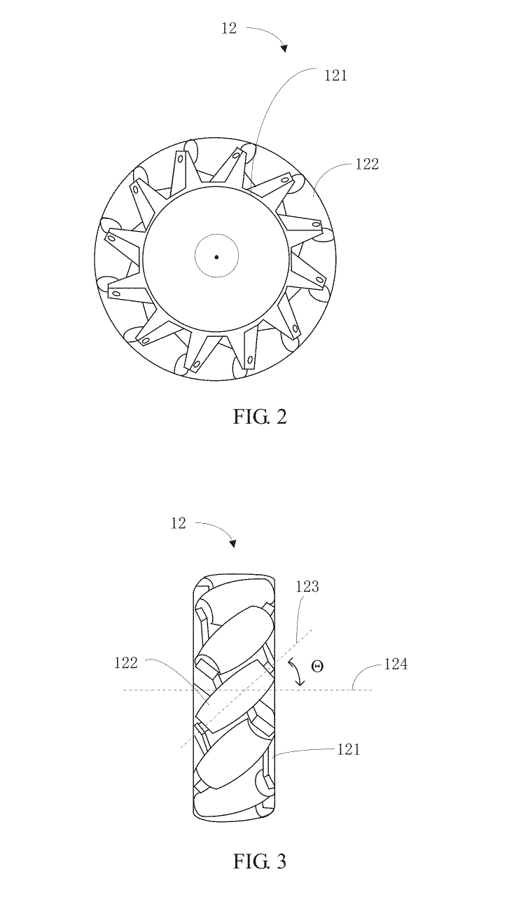

FIG. 2 is a front view illustrating a movable wheel of a medical apparatus according to an example of the present disclosure; and FIG. 3 is a left view illustrating a movable wheel of a medical apparatus according to an example of the present disclosure. The movable wheel 12 as shown in FIG. 2 and FIG. 3 may include a hub 121 and a plurality of driven wheels 122 disposed at the periphery of the hub. An axis 123 of one of the driven wheels 122 and an axis 124 of the hub 121 form an included angle Θ, which may range from 15 to 75 degrees. In an example, the included angle Θ may be, but is not limited to, 45 degrees.

In an example, the driven wheels 122 may be uniformly disposed at the periphery of the hub 121. In an example, the axis 123 of each of the driven wheels 122 and the axis 124 of the hub 121 form a same included angle, and a distance between the axes 123 of every two adjacent driven wheels 122 is identical.

The hub 121 may be controlled to rotate so as to drive the driven wheels 122 to rotate. In an example, when the hub 121 is controlled to rotate, the driven wheels 122 may be driven to rotate due to friction between contact parts of an external surface of the periphery of the hub 121 and external surfaces of the driven wheels 122. In this way, the driven wheels 122 may roll relative to the ground, so that the movable wheel 12 may move relative to the ground. Rotating speed and rotating direction of the hub 121 may be adjusted, so that rotating speed and rotating direction of the movable wheel 12 may be adjusted. The rotating direction of the hub 121 may include forward rotation and backward rotation. The rotating direction of the movable wheel 12 may include forward rotation and backward rotation.

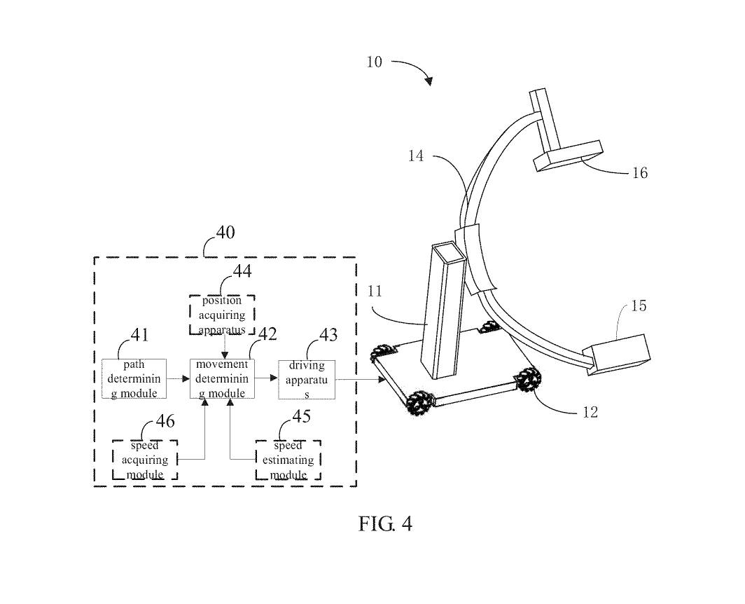

FIG. 4 illustrates a module block diagram of the medical apparatus 10 shown in FIG. 1 according to an example of the present disclosure. The medical apparatus 10 may include a movement controller 40. In an example, the movement controller 40 may be disposed within the controller 13 shown in FIG. 1. In another example, part of modules/devices of the movement controller 40 may be disposed within the controller 13, and remaining part of modules/devices may be disposed independent of the controller 13.

The movement controller 40 may be configured to control rotation of the plurality of movable wheels 12, and further control a moving direction and speed of the gantry 11 by controlling the rotation of the plurality of movable wheels 12. On one hand, the movement controller 40 may control the gantry 11 to move toward the scanning bed 17 or adjust the position of the gantry 11 around the scanning bed 17 so that a subject placed on the scanning bed 17 may be located within a scanning field of view. Thus, the X-ray source 15 and the detector 16 supported by the support arm 14 may scan a region of interest of the subject based on control signals received from the controller 40.

On the other hand, the movement controller 40 may also control the gantry 11 to move away from the scanning bed 17 after a scan is completed, for example, control the gantry 11 to move to a parking position of the gantry 11 when the medical apparatus 10 does not work, where the parking position may be reasonably set according to a site condition in a practical application.

Since the medical apparatus 10 herein is provided with the plurality of movable wheels 12 and the movement controller as mentioned above, the medical apparatus 10 may move to a target position more flexibly, rapidly and accurately, providing superior user experience for the medical personnel.

In an example, the movement controller 40 may include a path determining module 41, a movement determining module 42, and a driving device 43. The path determining module 41 may be configured to determine a moving path of the gantry 11. The movement determining module 42 may be configured to determine a rotating speed and a rotating direction of each movable wheel 12 according to the moving path. The driving device 43 may be configured to drive each movable wheel 12 to rotate according to the determined rotating speed and rotating direction for each movable wheel 12.

In an example, the path determining module 41 may plan a path for the gantry 11 to move from a start position to a target position based on the start position and the target position, and determine the path as the moving path of the gantry 11. For example, the path determining module 41 may plan a shortest path from the start position to the target position. In addition, the path determining module 41 may comprehensively plan a path based on conditions such as a site where the medical apparatus 10 is located and positions of other apparatuses or objects in the site. The start position may represent a current position of the gantry 11 and may also be the parking position of the gantry 11, a position of the gantry 11 when the medical apparatus 10 performs a current scan on a subject (hereinafter referred to as a current scanning position), or any position that the gantry 11 is to pass by between the parking position and the current scanning position. The start position of the gantry 11 may be obtained by detection. The target position may be the parking position of the gantry 11, a position of the gantry 11 when the medical apparatus 10 performs a subsequent scan on the subject (hereinafter referred to as a subsequent scanning position), or any position that the gantry 11 is to pass by between the parking position and the subsequent scanning position. The target position may be set according to a working condition requirement of the medical apparatus 10.

In an example, a plurality of moving paths may be preset for the gantry 11 according to information such as an actual structure of the site where the medical apparatus 10 is located and the position of the scanning bed 17, thereby adapting to different working conditions of the medical apparatus 10. The preset moving path may be for example a moving path of the gantry 11 from the current scanning position to the parking position and a moving path of the gantry 11 from the parking position to the subsequent scanning position. The path determining module 41 may select one of the plurality of preset moving paths as the moving path of the gantry 11 based on actual needs. In an example, a plurality of moving modes, such as a moving speed, a rotation manner, a linear movement, a curved movement, etc., may be preset for the gantry 11. The path determining module 41 may also be configured to determine a moving mode of the gantry 11. The preset moving paths and/or moving modes may be displayed on the display of the above controller 13, other display apparatuses (e.g. a control panel) or an independent display for selection by a user.

The movement determining module 42 may determine a rotating speed and a rotating direction for each movable wheel according to the moving path and the moving mode selected by the user, so that the driving device 43 may drive each movable wheel 12 to rotate according to the determined rotating speed and rotating direction.

The driving device 43 may be disposed at an axle of the movable wheel 12 (that is, axis of the hub 121) and the like. The driving device 43 may include a miniature motor. In an example, each movable wheel 12 may be provided with a driving motor for driving the hub 121 of the movable wheel 12 to rotate.

In another example, as shown in FIG. 4, the movement controller 40 may also include a position acquiring device 44. The functions of the path determining module 41, the movement determining module 42 and the driving device 43 may be referred to the above description and will not be repeated here. The position acquiring device 44 may be configured to acquire a position of the gantry 11 at a current moment. When a distance that the position of the gantry 11 at the current moment deviates from the moving path is greater than a distance threshold, the movement determining module 42 may adjust at least one of the rotating speed and the rotating direction of the movable wheel 12 based on the deviation distance of the gantry 11. Thus, the distance that the position of the gantry 11 at the current moment deviates from the moving path may be reduced, thereby improving the timeliness of arrival of the gantry at the target position.

In an example, the position acquiring device 44 may determine whether the distance that the position of the gantry 11 at the current moment deviates from the moving path is greater than the distance threshold. In an example, the position acquiring device 44 may determine the distance that the current position of the gantry 11 deviates from the moving path after acquiring the current position of the gantry 11 and send a signal of adjusting the rotating speed and/or the rotating direction of the movable wheels 12 to the movement determining module 42 when determining the deviation distance is greater than the distance threshold. The movement determining module 42 may re-determine the rotating speed and/or the rotating direction of the movable wheels 12 according to the distance that the current position of the gantry 11 deviates from the movable path after receiving the signal, thereby adjusting the movable wheels 12 to move at the re-determined rotating speed and in the re-determined rotating direction. In some cases, the movement determining module 42 may reduce the distance that the gantry 11 deviates from the moving path by adjusting the rotating speed of the movable wheels 12. In some cases, the movement determining module 42 may also reduce the distance that the gantry 11 deviates from the moving path by adjusting the rotating direction of the movable wheels 12. In other cases, the movement determining module 42 may also reduce the distance that the gantry 11 deviates from the moving path by adjusting the rotating speed and the rotating direction of the movable wheels 12. In some cases, the movement determining module 42 may adjust the rotating speeds and/or the rotating directions of all the movable wheels 12. However, in some cases, the movement determining module 42 may only adjust the rotating speeds and/or the rotating directions of part of the movable wheels 12.

In another example, the movement determining module 42 may determine whether the distance that the position of the gantry 11 at the current moment deviates from the moving path is greater than the distance threshold. In an example, after obtaining the position of the gantry 11 at the current moment, the position acquiring apparatus 44 may send the position of the gantry 11 to the movement determining module 42. The movement determining module 42 may then determine the distance that the gantry 11 deviates from the moving path. When determining that the distance that the gantry 11 deviates from the moving path is greater than the distance threshold, the movement determining module 42 may re-determine the rotating speeds and/or rotating directions of at least part of the movable wheels 12, so that the at least part of movable wheels may be adjusted to move at the re-determined rotating speeds and in the re-determined rotating directions.

The distance threshold may be an acceptable maximum error value that the gantry 11 deviates from a moving path, which may be set according to experience or practical application. The distance threshold may be preset, and may also be determined in the moving process of the gantry 11.

In an example, the position acquiring device 44 may include, but is not limited to, an inertial position acquiring device or a laser position acquiring device.

The laser position acquiring device may include a laser scanner, a laser reflecting plate, and a position determining module, where the laser scanner may be mounted on the gantry 11 and configured to emit a laser beam and the laser reflecting plate may be mounted in a region, for example, on a surrounding wall of the site where the medical apparatus 10 is located and configured to reflect a laser beam emitted by the laser scanner. When the gantry 11 moves, the position acquiring module 44 may determine a real-time position of the gantry 11 based on the laser beam emitted by the laser scanner in real time and the laser beam reflected by the laser reflecting plate. The laser position acquiring device may also acquire a moving direction of the gantry 11. For example, the laser position acquiring device may also determine the moving direction of the gantry 11 according to the laser beam emitted by the laser scanner in real time and the laser beam reflected by the laser reflecting plate.

The inertial position acquiring device may include a gyroscope, a positioning block and a position determining module, where the gyroscope may be mounted on the gantry 11 and the locating block may be mounted in a region, for example, on a wall of the site where the medical apparatus 10 is located. The gyroscope may emit a signal and the positioning block may feed back the signal emitted by the gyroscope. When the gantry 11 moves, the position acquiring module 44 may acquire the signal emitted by the gyroscope and the signal fed back by the positioning block, and determine the position of the gantry 11 according to the emitted signal and the fed-back signal. The inertial position acquiring device may also acquire the rotating direction of the gantry 11. For example, the inertial position acquiring apparatus may determine the moving direction of the gantry 11 according to the emitted signal and the fed-back signal.

In another example, as shown in FIG. 4, the movement controller 40 may also include a speed estimating module 45 and a speed acquiring module 46. The speed estimating module 45 may be configured to estimate a desired moving speed of the gantry 11 along the moving path. The speed acquiring module 46 may be configured to acquire the real-time speed of the gantry 11 at a current moment. When a difference between the real-time speed and the desired speed is greater than a speed difference threshold, the movement determining module 42 may adjust the rotating speed of the movable wheels 12 according to the difference between the real-time speed and the desired speed, thereby improving the timeliness of arrival of the gantry 11 at the target position.

In an example, the desired speed is a speed at which the gantry 11 is expected to move along the moving path determined by the path determining module 41. The speed estimating module 45 may estimate a desired moving speed of the gantry 11 according to the moving path determined by the path determining module 41 and in combination with an empirical value or a practical application. The speed estimating module 45 may also estimate the desired moving speed of the gantry 11 based on the rotating speed and the rotating direction of the movable wheel 12 determined by the movement determining module 42. The desired moving speed at which the gantry 11 moves along the determined moving path may be constant, and may also be variable. In an example, the speed acquiring module 46 may determine whether a difference between the real-time speed and the desired speed of the gantry 11 is greater than a speed difference threshold. In an example, after acquiring the real-time speed of the gantry 11 at the current moment, the speed acquiring module 46 may calculate a difference between the real-time speed and the desired speed, and may send a signal of adjusting the rotating speed of the movable wheels 12 to the movement determining module 42 when determining that the difference is greater than the speed difference threshold. In this case, the movement determining module 42 may re-determine the rotating speed of the movable wheels 12 based on the difference and control the movable wheels 12 to move based on the determined rotating speed.

In another example, the movement determining module 42 may determine whether a difference between the real-time speed and the desired speed of the gantry 11 is greater than the speed difference threshold. In an example, after the speed acquiring module 46 acquires the real-time speed of the gantry 11 at the current moment and the speed estimating module 45 determines the desired speed of the gantry 11, the movement determining module 42 may calculate a difference between the real-time speed and the desired speed of the gantry 11, and may re-determine the rotating speed of the movable wheels 12 according to the difference when determining that the difference is greater than the speed difference threshold.

The speed difference threshold may be an acceptable maximum error value between the actual speed and the desired speed of the gantry 11, which may be set according to experience or a practical application. The speed difference threshold may be preset, and may also be determined in a moving process of the gantry 11.

In an example, in the case that the movement controller 40 includes the position acquiring apparatus 44, the speed estimating module 45 and the speed acquiring module 46, during a moving process of the gantry 11, a motion trajectory of the gantry 11 may be adjusted in real time to be substantially consistent with a desired moving path, and the gantry 11 may also be adjusted to substantially move at a desired moving speed.

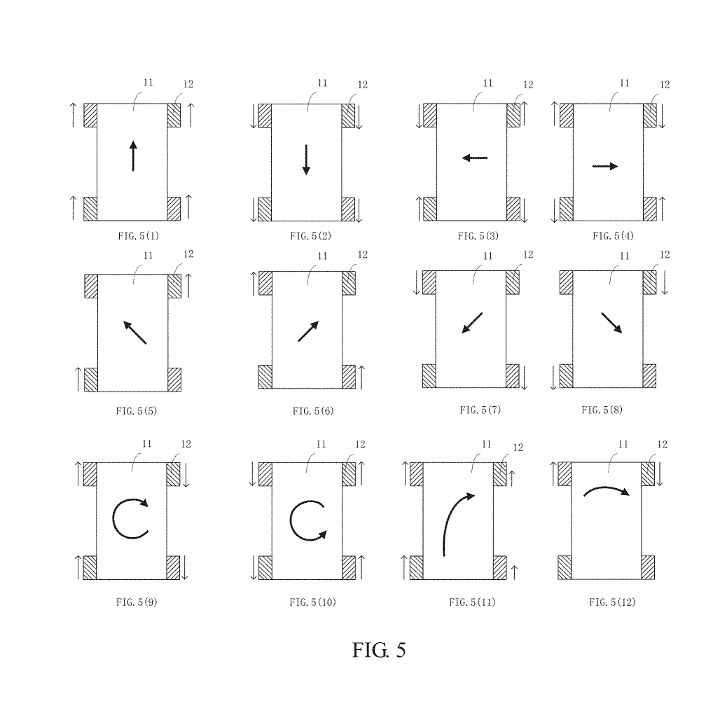

FIG. 5(1) to FIG. 5(12) are schematic diagrams illustrating a driving mode of controlling movement of the gantry 11. In the illustrated example, taking a plurality of movable wheels 12 being four mecanum wheels as an example, description will be made to a case that the movement controller 40 controls the four mecanum wheels to rotate so as to control the gantry 11 to move.

As shown in FIG. 5(1) to FIG. 5(12), when the four mecanum wheels rotate forward at a same speed and in a same direction as shown in FIG. 5(1), the movement of the four wheels combined may cause the gantry 11 to move forward along a straight line.

When the four mecanum wheels rotate backward at the same speed and in the same direction as shown in FIG. 5(2), the movement of the four wheels combined may cause the gantry 11 to move backward along a straight line.

When the four mecanum wheels rotate at the same speed with a left front wheel and a right rear wheel both rotating backward, and a left rear wheel and a right front wheel both rotating forward as shown in FIG. 5(3), the movement of the four wheels combined may cause the gantry 11 to move left along a straight line.

When the four mecanum wheels rotate at the same speed with the left front wheel and the right rear wheel both rotating forward, and the left rear wheel and the right front wheel both rotating backward as shown in FIG. 5(4), the movement of the four wheels combined may cause the gantry 11 to move right along a straight line.

When only the left rear wheel and the right front wheel of the four mecanum wheels are driven to rotate forward at the same speed and in the same direction as shown in FIG. 5(5), the movement of the four wheels combined may cause the gantry 11 to move in a left forward direction along a straight line.

When only the left front wheel and the right rear wheel of the four mecanum wheels are driven to rotate forward at the same speed and in the same direction as shown in FIG. 5(6), the movement of the four wheels combined may cause the gantry 11 to move in a right forward direction along a straight line.

When only the left front wheel and the right rear wheel of the four mecanum wheels are driven to rotate backward at the same speed and in the same direction as shown in FIG. 5(7), the movement of the four wheels combined may cause the gantry 11 to move in a left backward direction along a straight line.

When only the left rear wheel and the right front wheel of the four mecanum wheels are driven to rotate backward at the same speed and in the same direction as shown in FIG. 5(8), the movement of the four wheels combined may cause the gantry 11 to move in a right backward direction along a straight line.

When the four mecanum wheels rotate at the same speed with two left wheels rotating forward, and two right wheels rotating backward as shown in FIG. 5 (9), the movement of the four wheels combined may cause the gantry 11 to move clockwise.

When the four mecanum wheels rotate at the same speed with the two left wheels rotating backward, and the two right wheels rotating forward as shown in FIG. 5(10), the movement of the four wheels combined may cause the gantry 11 to move counterclockwise.

When the two left wheels of the four mecanum wheels rotate at a same speed and the two right wheels rotate at a same speed where the rotating speed of the two left wheels is greater than that of the two right wheels, and the four wheels rotate forward as shown in FIG. 5(11), the movement of the four wheels combined may cause the gantry 11 to move in a right forward direction along a curve shown in FIG. 5(11).

When only two front wheels of the four mecanum wheels are driven to rotate at a same speed with a left front wheel rotating forward, and a right front wheel rotating backward as shown in FIG. 5(12), the movement of the four wheels combined may cause the gantry 11 to move in a right transverse direction along a curve shown in FIG. 5(12).

The rotating speed and the rotating direction of the gantry 11 provided with four mecanum wheels depend on the magnitude and direction of a driving force used by the driving device 43. FIG. 5(1) to FIG. 5(12) only enumerate part of driving modes. In addition, more types of motion trajectories may be realized based on combinations of the rotating speeds and the rotating directions of the four mecanum wheels, so that the gantry 11 may move in different directions or along different motion trajectories according to actual requirements.

It is to be noted that, a gantry provided with other types of movable wheels may also have driving modes and motion trajectories similar to those of the above gantry 11 with the mecanum wheels. When a number of the plurality of movable wheels 12 is another number, rotating speeds and rotating directions of these movable wheels 12 may be combined with reference to the driving modes as described above, so that the gantry 11 may move along different trajectories, which will not be described here one by one.

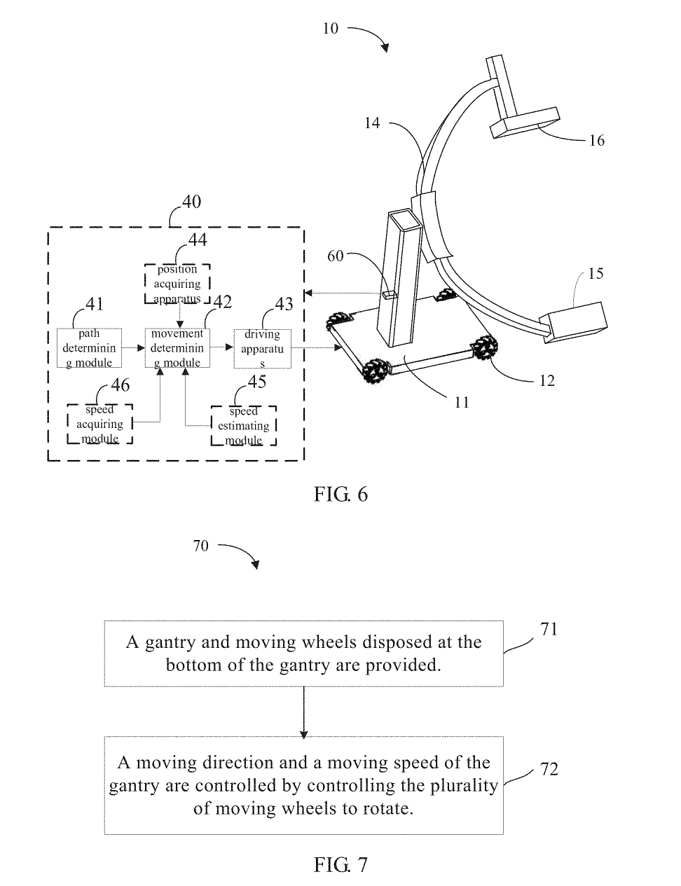

FIG. 6 is a module block diagram illustrating a medical apparatus 10 according to another example of the present disclosure. The medical apparatus 10 shown in FIG. 6 is similar to the medical apparatus 10 shown in FIG. 4. Compared with the medical apparatus 10 shown in FIG. 4, the medical apparatus 10 shown in FIG. 6 may further include an anti-collision device 60.

The anti-collision device 60 may be configured to detect a distance between an object and the gantry 11. In this way, when the anti-collision device 60 detects that the distance between the object and the gantry 11 is smaller than a safe distance, the movement controller 40 may adjust the movements of the movable wheels 12. The object may be a wall, some devices of the medical apparatus 10, other medical apparatuses, or other articles in the site where the medical apparatus 10 is located. In some cases, the object may also be a subject, an operator, or the like.

In an example, the anti-collision device 60 may include one or more anti-collision sensors, which may be disposed in any position on the gantry 11. In an example, the anti-collision sensor may be disposed at the bottom of the gantry 11. The anti-collision sensor may be configured to detect a distance between the object and the gantry 11.

In an example, the anti-collision device 60 may also include a distance comparing module. The distance comparing module may be disposed within the anti-collision sensor or independently disposed in any position of the gantry 11. The distance comparing module may receive a distance between the object and the gantry 11 detected by the one or more anti-collision sensors, and send a signal to the movement determining module 42 when it is determined that the distance between the object and the gantry 11 is smaller than the safe distance, so that the movement determining module 42 may adjust the movements of the movable wheels 12.

In another example, the movement determining module 42 may determine whether the detected distance between the object and the gantry 11 is smaller than the safe distance. The anti-collision sensor may send the detected distance between the object and the gantry 11 to the movement determining module 42. The movement determining module 42 may then compare the distance with the safe distance, and adjust the movements of the movable wheels 12 when it is determined that the distance between the object and the gantry 11 is smaller than the safe distance.

The safe distance may be an acceptable minimum distance between an object and the gantry 11. In an example, the safe distance may be an acceptable minimum distance between an object and an anti-collision sensor on the gantry 11.

It is to be noted that, in an example, adjusting the movement of the movable wheels 12 may refer to stopping the movement of the movable wheels 12 so as to stop the gantry 11.

In another example, adjusting the movement of the movable wheels 12 may refer to changing the rotating direction and/or rotating speed of the movable wheels 12 so as to allow the gantry 11 to avoid an object.

In this example, by adding the above anti-collision apparatus 60, the gantry 11 can be effectively prevented from hitting the personnel, the scanning bed 17 or other apparatuses during a moving process; meanwhile, damage to the medical apparatus 10 arising from collision with other objects may also be reduced.

In an example, the anti-collision device 60 may also be mounted on the support arm 14, so that risks that the support arm 14 collides with the personnel, the scanning bed 17 or other apparatuses during a rotating process may be reduced.

FIG. 7 is a flowchart illustrating a method of controlling movement of a medical apparatus according to an example of the present disclosure. The method of controlling movement of a medical apparatus may include block 71 and block 72.

At block 71, a gantry and a plurality of movable wheels disposed at the bottom of the gantry are provided, where each of the movable wheels includes a hub and a plurality of driven wheels disposed on the hub; and an axis of the driven wheel and an axis of the hub form an included angle.

A number of the movable wheels 12 at the bottom of the gantry may be set according to a practical application. In an example, the number of the movable wheels 12 may be an even number equal to or greater than 4, such as 6 or 8. The gantry may be more easily controlled by setting an even number of movable wheels 12. In an example, the number of the movable wheels 12 may be an even number equal to or greater than 6 such as 10 or 12. With the number of movable wheels being even and greater than 6, the gantry 11 may be better supported, so that the gantry 11 may be more stable in a moving process. The movable wheels 12 may include, but are not limited to, mecanum wheels.

In an example, the medical apparatus may be an angiography apparatus, for example, an angiography machine. Of course, the medical apparatus may also be other apparatuses for assisting diagnosis and treatment.

At block 72, a moving direction and a moving speed of the gantry are controlled by controlling the plurality of movable wheels to rotate, where the hub of each movable wheel is controlled to rotate so as to drive the driven wheels to rotate, thereby controlling the rotating speed and the rotating direction of the movable wheel.

According to the method of controlling movement of a medical apparatus provided in the present disclosure, the medical apparatus may move to a target position more flexibly, quickly and accurately.

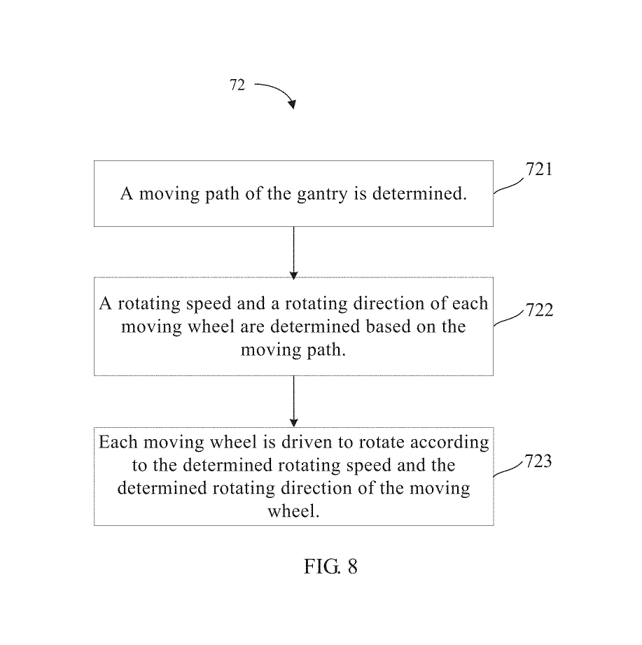

FIG. 8 is a flowchart illustrating a method of controlling movement of a movable wheel at block 72 according to an example of the present disclosure. The method of controlling movement of a movable wheel may include block 721 to block 723.

At block 721, a moving path of the gantry is determined.

In an example, a start position and a target position of the gantry may be determined according to a requirement, so that a path, i.e., the moving path of the gantry, along which the gantry moves from the start position to the target position may be determined. In an example, one or more moving paths may be preset and one of the moving paths may be determined as the moving path of the gantry.

At block 722, a rotating speed and a rotating direction of each movable wheel are determined based on the moving path.

At block 723, each movable wheel is driven to rotate according to the determined rotating speed and the determined rotating direction of the movable wheel.

In an example, the method of controlling movement of a movable wheel may also include: acquiring a position of the gantry 11 at a current moment; and adjusting at least one of the rotating speed and the rotating direction of the movable wheel 12 when a distance that the position of the gantry 11 at the current moment deviates from the moving path is greater than a distance threshold. Thus, the distance that the position of the gantry 11 deviates from the moving path may be reduced, thereby effectively improving the timeliness of arrival of the gantry at the target position. The distance threshold may be an acceptable maximum error value that the gantry deviates from the moving path, and may be set according to experience or practical application. The distance threshold may be preset, and may also be determined during a moving process of the gantry.

In an example, the method of controlling movement of a movable wheel may also include: estimating a desired moving speed at which the gantry moves along the moving path; acquiring a real-time speed of the gantry; and adjusting the rotating speed of the movable wheel when a difference between the real-time speed and the desired speed is greater than a speed difference threshold. In this way, the timeliness of arrival of the gantry at the target position may be improved.

In an example, the method of controlling movement of a movable wheel may also include: detecting a distance between an object and the gantry; and adjusting the movement of the movable wheels when the distance between the object and the gantry is smaller than a safe distance. Thus, the gantry may be prevented from hitting the personnel, the scanning bed or other apparatuses during a moving process; meanwhile, damage to the medical apparatus arising from collision with other objects may also be reduced.

The safe distance may be a distance threshold from the gantry, and may also be a distance value between a boundary of a safe region and the gantry. For example, the safe region may be a circular region defined with the gantry as a center and the safe distance as a radius.

Actions of the method 70 of controlling a medical apparatus to move are illustrated in the form of modules. The sequence of the modules shown in FIGs and the partition of actions of the modules are not limited to the illustrated example. For example, the modules can be executed in a difference sequence; the action in one module may be combined with the action in another module or split into a plurality of modules. In some examples, other blocks may also be included before, between or after the illustrated blocks of the method 70 of controlling a medical apparatus to move.

With respect to method examples, since the method examples substantially correspond to the apparatus examples, a reference may be made to part of the description of the apparatus examples for the related part. The above method may be implemented by the apparatuses as discussed herein, and may also be implemented by other apparatuses. The method examples and the apparatus examples complement each other.

The foregoing are merely descriptions of preferred examples of the present disclosure and not intended to limit the present disclosure. Any modifications, equivalent substitutions, adaptations and the like made within the spirit and the principles of the present disclosure shall be encompassed in the scope of protection of the present disclosure.

What is claimed is:

1. A medical apparatus, comprising:
a gantry;
a plurality of movable wheels disposed at a bottom of the gantry, each of the plurality of movable wheels comprises:
a hub; and
a plurality of driven wheels disposed at a periphery of the hub, wherein an axis of each of the driven wheels and an axis of the hub form an included angle, and rotation of the hub is capable of causing rotation of the plurality of driving wheels; and
a movement controller configured to control a moving direction and a moving speed of the gantry by controlling rotation of the plurality of movable wheels.

2. The medical apparatus of claim 1, wherein the plurality of movable wheels comprise mecanum wheels.

3. The medical apparatus of claim 1, wherein a number of the movable wheels is an even number equal to or greater than 4.

4. The medical apparatus of claim 1, wherein the movement controller comprises:
a path determining module configured to determine a moving path of the gantry;
a movement determining module configured to determine a rotating speed and a rotating direction of each of the plurality of movable wheels according to the moving path; and
a driving device configured to drive each of the plurality of movable wheels to rotate according to the determined rotating speed and the determined rotating direction of each of the plurality of movable wheels.

5. The medical apparatus of claim 4, wherein the movement controller further comprises:
a position acquiring device configured to acquire a position of the gantry at a current moment as a current position of the gantry.

6. The medical apparatus of claim 5, wherein the movement determining module is further configured to adjust at least one of the rotating speed and the rotating direction of at least some of the plurality of movable wheels when a distance that the current position of the gantry deviates from the moving path is greater than a distance threshold.

7. The medical apparatus of claim 4, wherein the movement controller further comprises:
a speed estimating module configured to estimate a desired moving speed of the gantry along the moving path; and
a speed acquiring module configured to acquire a real-time speed of the gantry.

8. The medical apparatus of claim 7, wherein the movement determining module is further configured to adjust the rotating speed of at least some of the plurality of movable wheels when a difference between the real-time speed and the desired speed is greater than a speed difference threshold.

9. The medical apparatus of claim 1, further comprising:
an anti-collision apparatus configured to detect a distance between an object and the gantry.

10. The medical apparatus of claim 9, wherein the movement controller adjusts movement of at least some of the plurality of movable wheels when the anti-collision apparatus detects that the distance between the object and the gantry is smaller than a safe distance.

11. A method of controlling a medical apparatus to move, comprising:
disposing a plurality of movable wheels at a bottom of a gantry of the medical apparatus, wherein each of the plurality of movable wheels comprises a hub, and a plurality of driven wheels disposed at a periphery of the hub; an axis of each of the driven wheels and an axis of the hub form an included angle, and rotation of the hub is capable of causing rotation of the driven wheels; and controlling a moving direction and a moving speed of the gantry by controlling respective hub of one or more movable wheels to rotate.

12. The method of claim 11, wherein the plurality of movable wheels comprise mecanum wheels.

13. The method of claim 11, wherein a number of the movable wheels is an even number equal to or greater than 4.

14. The method of claim 11, wherein controlling the respective hub of one or more movable wheels to rotate comprises:
determining a moving path of the gantry;
determining a rotating speed and a rotating direction of each of the plurality of movable wheels according to the moving path; and
driving each of the plurality of movable wheels to rotate according to the determined rotating speed and rotating direction of each of the plurality of movable wheels.

15. The method of claim 14, further comprising:
acquiring a position of the gantry at a current moment; and
adjusting at least one of the rotating speed and the rotating direction of at least some of the plurality of movable wheels when a distance that the position of the gantry at the current moment deviates from the moving path is greater than a distance threshold.

16. The method of claim 14, further comprising:
estimating a desired moving speed of the gantry along the moving path;
acquiring a real-time speed of the gantry; and
adjusting the rotating speed of at least some of the movable wheels when a difference between the real-time speed and the desired speed is greater than a speed difference threshold.

17. The method of claim 11, further comprising:
detecting a distance between an object and the gantry; and
adjusting movement of at least some of the plurality of movable wheels when detecting that the distance is smaller than a safe distance.
